(19) United States
(12) Patent Application Publication
(10) Pub. No.: US 2008/0096268 A1
(43) Pub. Date: Apr. 24, 2008

(54) Biotrickling filter packing material and systems and methods of using same to remove odour causing compounds from waste gas streams

(75) Inventors: Brian P. Herner, Georgetown (CA); Scott Shugao Zhang, Guelph (CA)

Correspondence Address: FASKEN MARTINEAU DUMOULIN LLP, 4200 TORONTO DOMINION BANK TOWER, BOX 20 TORONTO-DOMINION CENTRE, TORONTO, ON, M5K 1N6, US

(73) Assignee: Biorem Technologies Inc.

(21) Appl. No.: 11/583,783

(22) Filed: Oct. 20, 2006

(51) Int. Cl. A61L 9/01

(52) U.S. Cl. 435/266

(57) ABSTRACT

The present invention relates to biotrickling filter systems and the packing material employed in such systems, as well as, methods of using same to remove odour causing compounds from waste gas streams. The packing material for the biotrickling filter system includes a plurality of expanded glass granules. When used in a biotrickling filter system, the packing material is highly efficient at removing from waste gas streams hydrogen sulfide at high concentrations in low empty bed residence times.

### FIELD OF THE INVENTION

The present invention relates to biotrickling filter systems and the packing material employed in such systems, as well as, methods of using same to remove odour causing compounds from waste gas streams.

### BACKGROUND OF THE INVENTION

Wastewater treatment plants, rendering plants, food processing, flavour manufacturing and composting facilities generate significant volumes of waste gas streams containing odour causing compounds. Prior to exhausting these waste gas streams into the environment, these facilities are required to treat the contaminated air to remove odour causing compounds such as hydrogen sulfide and volatile organic compounds (VOC). Traditionally, these waste gas streams have been treated using physico-chemical methods involving one or more of the following techniques: condensation, adsorption, absorption, chemical scrubbing and oxidation. Chemical scrubbers have been used to reduce odour in waste gas streams to very low levels. However, such scrubbers tend to be expensive to deploy and operate, as they require safe storage, metering, and control equipment. Moreover, such chemical scrubbers employ hazardous chemicals such as caustic and chlorine or sodium hypochlorite. The treatment of hydrogen sulfide contaminated air tends to require relatively large amounts of these chemicals thus adding significantly to the operational cost of the scrubber.

In light of the foregoing, other treatment techniques have been considered. One such treatment involves using biofiltration techniques and systems to treat the waste gas streams. Notably, biotrickling filters have been used to remove odour causing compounds from waste gas streams.

In a typical biotrickling filter, the waste gas stream is passed through the inert packing material of a packed bed while a recirculated liquid is trickled therethrough. Mass transfer of the contaminants occurs from the gaseous phase to the liquid phase as the contaminants are solubilized in the trickling fluid. Once in the liquid phase, the contaminants are adsorbed to the packing material. Thereafter, the contaminants are biodegraded by microbial cultures growing within the biofilm that is supported on the packing material. The overall efficiency of the process is determined by the relative rates of phase transfer, adsorption and the biological reactions. These rates may be optimized by careful selection of the packing material for the biotrickling filter.

Accordingly, selecting the appropriate packing material is critical to ensure proper functioning of the biotrickling filter. While the packing material serves multiple purposes, its most important function tends to be providing contact between the gas-phase contaminants and the active microbial colonies immobilized on the biofilm. In considering the suitability of a material for use as a biotrickling filter media, the following factors are considered to be desirable: the ability to support bacterial growth, large surface area, structural integrity (i.e. resistance to compaction), high porosity, low bulk density, low chemical reactivity, pH buffering capacity, good adsorption properties, sufficient water retention capability, non-biodegradability and cost effectiveness.

Different packing materials have been used in previous biotrickling filter applications. Examples of such packing materials include lava rocks, extruded plastic packing (such as Raschig rings and Berl saddles), fibres, glass beads, porous ceramic cylinders and open-cell foams. However, as explained in greater detail below, each of these packing materials has experienced one drawback or another in the field leaving a need for an improved packing material.

Lava rocks tend to be generally available and inexpensive. They tend to exhibit superior resistance to acid and compaction and have rough surfaces that tend to promote biofilm attachment. However, they tend to have a high bulk density that limits the depth to which the packing bed may be constructed without expensive reinforced structures. The energy required to force the waste gas stream through the bed of lava rock tends to be quite high. As a result, biotrickling filters that employ rock media tend to be designed to accommodate only very low gas velocities and accordingly, have large footprints.

Extruded plastic packing tends to be acid resistant and have a low bulk density. Additionally, this packing tends to experience a low gas pressure drop and promote good liquid distribution. However, its resistance to compaction tends to be limited. Moreover, its surface tends to be generally unsuitable for biofilm attachment and growth resulting in relatively low bacterial populations per unit volume. In some cases, the treatment of contaminated air in biotrickling filters employing plastic packing material may require residence times in the order of several minutes.

Fibrous mesh pads tend to have a high surface area and a low bulk density. However, they have a tendency to compact due to the weight of the biomass growth eventually leading to increased gas-phase pressure drop and clogging. Moreover, fibrous mesh pads tend not to distribute trickling water evenly.

Glass beads tend to be suitable carriers for microorganisms and tend to tolerate acid conditions prevailing in the biotrickling filter. Advantageously, water soluble sulfur compounds formed during the treatment of the contaminated may be easily washed away from the surface of the glass beads. Glass beads, however, have very little porosity and tend not to retain moisture. Moreover, other packing materials, such as ceramics, offer larger surface areas which leads to more active microbial growth and improved contact time between the contaminated gas and fixed biofilm. On the other hand, ceramics tend to be very expensive to manufacture and may be difficult to source.

Open-cell polymeric foams have been used to some advantage as a packing material in biotrickling filters. More specifically, biotrickling filters with polymeric foam media have achieved high removal efficiencies for hydrogen sulfide and volatile organic compounds (VOC). This is due to their high surface area, high porosity and low bulk density, as well as their ability to retain biomass growth and their superior water retention capabilities. However, the weight of moist media limits the depth that can be used without excessive compaction. Over time, polymeric foam begins to compact due to biomass growth, resulting in lower void fractions and eventually high gas-phase pressure drop. Furthermore, gas channelling has been frequently observed.

In light of the foregoing, it would be desirable if a packing material having improved resistance to compaction, clogging, and excessive gas-phase pressure drop could be provided for use with a biotrickling filter. It would be further advantageous if a biotrickling filter using such a packing material were capable of achieving higher removal rates of hydrogen sulfide at greater concentrations with lower empty bed residence times (EBRT) than conventional biotrickling filters.

### SUMMARY OF THE INVENTION

In accordance with a broad aspect of an embodiment of the present invention, there is provided a method for removing odour causing compounds from a waste gas stream. The method includes providing a biotrickling filter system having a packing material. The packing material includes a plurality of expanded glass granules. The waste gas stream is urged to flow through the packing material of the biotrickling filter system. Additionally, a liquid is urged to trickle through the packing material. The method further includes collecting the trickling liquid in a sump and recirculating the trickling liquid from the sump into the biotrickling filter system for reuse. In another feature, the expanded glass granules are composed primarily of silica and alkali oxides and the alkali oxides are predominantly sodium oxide. In yet another feature, each expanded glass granule measures between 4 mm and 100 mm. More preferably, each expanded glass granule measures between 6 mm and 20 mm. Additionally, each expanded glass granule has a generally spherical shape.

In still a further feature, the step of urging the waste gas stream to flow through the packing material of the biotrickling filter system, includes forcing the waste gas stream to flow downwardly through the packing material. Alternatively, the step of urging the waste gas stream to flow through the packing material of the biotrickling filter system, includes forcing the waste gas stream to flow upwardly through the packing material. In another feature, the flow of the waste gas stream and the flow of the trickling liquid are one of counter-current and co-current.

In yet another feature, the step of urging a liquid to trickle through the packing material includes, distributing the liquid onto the surface of the packing material. The trickling liquid includes effluent and recirculated trickling liquid. Additionally, the trickling liquid includes a nutrient solution containing at least one of phosphorus, nitrogen and potassium.

In an additional feature, the odour causing compounds include hydrogen sulfide and volatile organic compounds.

In another broad aspect of an embodiment of the present invention, there is provided a biotrickling filter system. The biotrickling system includes a housing, an inlet connected to the housing for receiving contaminated air and an outlet connected to the housing for exhausting cleaned air. Also provided, is a packing bed located within the housing between the inlet and the outlet The packing bed has a packing material through which the contaminated air is forced to flow. The packing material includes a plurality of expanded glass granules. The biotrickling filter system further includes: means for drawing the contaminated air from the inlet into the packing bed and through the packing material, means for distributing a liquid onto the surface of the packing material for trickling therethrough, a sump disposed below the packing bed for collecting the trickling liquid, and means for recirculating the trickling fluid through the biotrickling filter. The liquid recirculating means is operatively connected to the sump and the liquid distributing means.

In still another broad aspect of an embodiment of the present invention, there is provided a method for removing odour causing compounds from a waste gas stream. The method includes providing a bioscrubber having a packing material. The packing material includes a plurality of expanded glass granules. The waste gas stream is urged to flow through the packing material of the bioscrubber. Additionally, a liquid is urged to trickle through the packing material. The method further includes collecting the trickling liquid in a sump and recirculating the trickling liquid from the sump into the bioscrubber for reuse. In an additional feature, the odour causing compounds include hydrogen sulfide and volatile organic compounds.

In yet another broad aspect of an embodiment the present invention, there is provided a bioscrubber. The bioscrubber includes a housing, an inlet connected to the housing for receiving contaminated air, and an outlet connected to the housing for exhausting cleaned air. Also provided is a packing bed located within the housing between the inlet and the outlet. The packing bed has a packing material through which the contaminated air is forced to flow. The packing bed defines an air phase reactor wherein contaminants in the contaminated air may undergo phase transfer from the gas phase to the liquid phase. The packing material includes a plurality of expanded glass granules. The method further includes: means for drawing the contaminated air from the inlet into the packing bed and through the packing material, means for distributing a liquid onto the surface of the packing material for trickling therethrough and a sump disposed below the packing bed for collecting the trickling liquid. The sump defines a liquid phase reactor wherein contaminants in the tricking fluid are biodegraded by suspended microbial cultures. Further provided, is means for recirculating the trickling fluid through the biotrickling filter. The liquid recirculating means is operatively connected to the sump and the liquid distributing means.

### BRIEF DESCRIPTION OF THE DRAWINGS

The embodiments of the present invention shall be more clearly understood with reference to the following detailed description of the embodiments of the invention taken in conjunction with the accompanying drawings, in which:

FIG. 1 is a simplified illustration of a biotrickling filter system provided with a packing material in accordance with an embodiment of the present invention;

FIG. 2 is a conceptual illustration of a granule of the packing material of the biotrickling filter shown in accordance with an embodiment of the present invention;

FIG. 3 is a graphical representation showing the hydrogen sulfide removal efficiency of a biotrickling filter system having the packing material provided in accordance with an embodiment of the present invention, plotted against the empty bed residence times (EBRT) at specified hydrogen sulfide concentrations in the waste gas stream;

FIG. 4 is a graphical representation of the required EBRT vs. hydrogen sulfide concentrations in the waste gas stream for specified removal efficiencies, for a biotrickling filter system having the packing material provided in accordance with an embodiment of the present invention; and

FIG. 5 is a simplified illustration of a bioscrubber provided with a packing material in accordance with an embodiment of the present invention.

### DETAILED DESCRIPTION OF EMBODIMENTS OF THE INVENTION

The description which follows, and the embodiments described therein are provided by way of illustration of an example, or examples of particular embodiments of principles and aspects of the present invention. These examples are provided for the purposes of explanation and not of limitation, of those principles of the invention. In the description that follows, like parts are marked throughout the specification and the drawings with the same respective reference numerals.

In the following specification, the terms “biotrickling filter” and “biotrickling filter system” refer to a system having packing material through which a liquid is recirculated or trickled and wherein the contaminants in a waste gas stream urged to flow therethrough are biodegraded primarily by the action of microorganisms in a fixed biofilm formed on the packing material. The term “bioscrubber”, “bioscrubbing filter” and “bioscrubbing filter system” refer to a system having packing material through which a liquid is flowed and wherein the contaminants in a waste gas stream urged to flow therethrough are biodegraded primarily by the action of suspended microbial cultures in a liquid holding tank. The term “packing material” refers to the media or packing material used in the filter beds of such biotrickling filter systems or bioscrubbers. Furthermore, the term “contaminants” or “air contaminants” refer to chemical compounds present in waste gas streams and includes, but is not limited to, sulfur-based compounds, such as hydrogen sulfide (“H₂S”), organic sulfides, reduced sulfur compounds, for instance, methyl mercaptan, dimethyl sulfide and dimethyl disulfide, and volatile organic compounds (“VOC”), such as aliphatic and aromatic compounds. Further, the terms “contaminated air stream” or “waste gas stream” refer to a flow of air/gas that contains contaminants.

Biotrickling Filter System

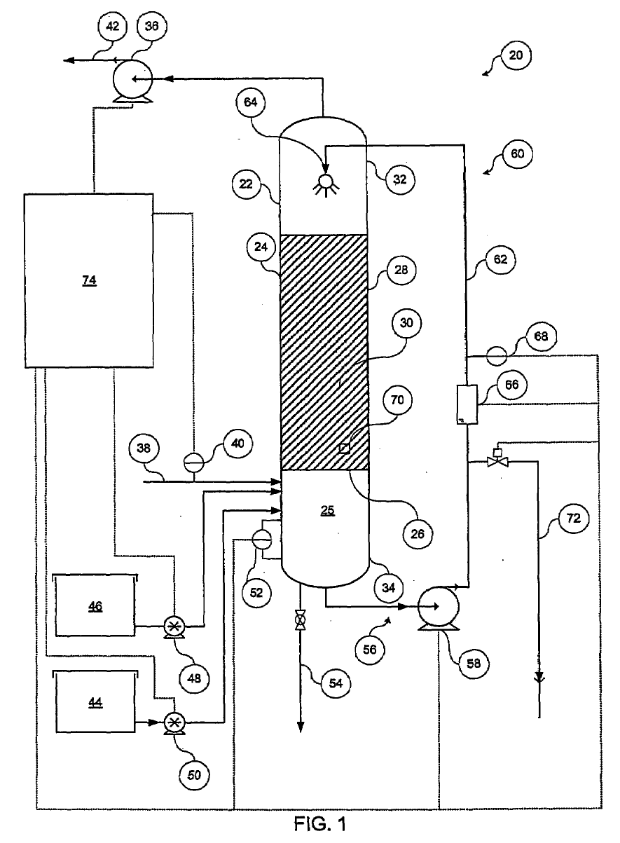

FIG. 1 shows a simplified illustration of a biotrickling filter system 20 according to an embodiment of the invention. The biotrickling filter system 20 includes a housing 22 that encloses a packing bed 24 and a sump or liquid reservoir 25. In this embodiment, the housing 22 is constructed of fibreglass-reinforced plastic (FRP) selected for its superior corrosion resistance to acids and its structural strength. However, in alternative embodiments, other materials exhibiting these characteristics could be used to similar advantage.

The packing bed 24 is carried above the sump 25 and has a vented support base 26 upon which rests a column 28 of packing material 30. The column 28 is disposed between the top and bottom ends 32 and 34 of the housing 22. A blower or fan 36 is provided to draw the contaminated air stream from a waste gas inlet 38 and through the packing bed 24. The waste gas inlet 38 is connected to housing 22 adjacent the bottom end 34 of the housing 22. A gas flow sensor 40 positioned within the waste gas inlet 38 measures the flow of contaminated air entering the biotrickling filter system 20.

In this embodiment, the biotrickling filter system 20 is operated as a counter-current system with the contaminated air stream being urged to flow upward through the packing bed 24 in a direction opposite to that of the trickling liquid. It will, however, be appreciated that in an alternative embodiment, the biotrickling filter could operate as a co-current system.

An outlet 42 located near the top end of the 32 of the housing 22 permits a cleaned air stream to exit the housing 22 following treatment in the packing bed 24. Optionally, a mist eliminator (not shown) positioned upstream of the outlet 42 could also be provided to remove fine liquid droplets from the cleaned air stream.

The sump 25 holds the trickling liquid which will be used in the biotrickling filter system 20. The trickling liquid may include effluent and/or freshwater and recirculated water and may be supplemented with a nutrient solution containing minerals such as nitrogen, phosphorus and potassium, and/or other additives such as liquid buffers for adjusting the pH of the packing material 30. In this embodiment, the effluent and nutrient solution are drawn into the sump 25 from dedicated effluent tank 44 and nutrient tank 46 using pumps 48 and 50, respectively. The effluent may be partially treated wastewater containing some residual nutrients. In other embodiments, the effluent may be provided by a supply line connected to a wastewater treatment plant. A liquid level sensor 52 measures the level of the trickling liquid in the sump 25. Some or all of the trickling fluid may be drained from the sump 25 through a first manually actuated drain line 54.

A liquid recirculation system 56 is connected to the sump 25. It includes a recirculation pump 58 and liquid distribution means 60. The recirculation pump 58 is operable to draw from the sump 25 the trickling liquid for delivery to the packing bed 24. The liquid distribution means 60 includes a feed line 62 with a plurality of nozzles 64 for continuously or intermittently spraying/trickling liquid onto the packing material 30. A liquid flow sensor 66 and a pH sensor 68 measure the flow rate and pH of the trickling liquid flowing through the feed line 62. Further provided, is a temperature sensor 70 to monitor the temperature of the packing material. Other sensors could also be provided for instance, to monitor the need for further nutrients. The liquid recirculation system 56 has a second drain line 72 for periodically purging the trickling fluid.

The biotrickling filter system 20 further includes a control system 74 that governs the operation of the system. The control system 74 communicates with (i.e. receives input signals from, and transmits output signals to,) the various sensors 40, 52, 66, 68 and 70 and other system equipment and can actuate the blower 36, the pumps 48, 50 and 58, the drain lines 54 and 72 and the liquid recirculation system 56 to ensure proper operation of the system.

Packing Material

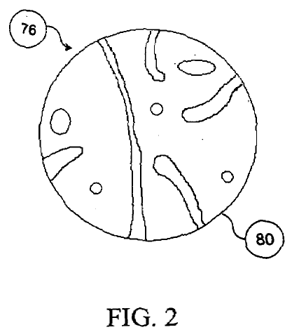

Within the biotrickling filter system 20, the packing material 30 is provided to remove contaminants from the contaminated air stream received within the housing 22. FIG. 2 conceptually illustrates a granule, bead or pellet 76 of the packing material according to an embodiment of the invention. Each granule 76 provides a surface area upon which may be supported the biofilm containing the microorganisms required to biodegrade the contaminants. Each granule 76 is an expanded glass granule 80 and is stable, inorganic non-reactive, non-flammable, non-toxic, non-odorous, non-biodegradable and acid resistant. In addition, the expanded glass granule 80 tends to be relatively hard and rigid which allows it to better resist warping or compaction from biomass growth and avoid high-gas phase pressure drop that may adversely impact on biotrickling filter performance. By virtue of its relatively high porosity, the expanded glass granule 80 tends to exhibit excellent moisture retention properties and has a relatively low bulk density. The expanded glass granule 80 is primarily composed of silica and alkali oxides (i.e. predominantly sodium oxide (Na₂O) and to a lesser extent, potassium oxide (K₂O)) with the remainder being composed of calcium oxide (CaO), alumina (Al₂O₃) and magnesium oxide (MgO). The chemical composition of the expanded glass granule 80 of this embodiment is set out below:

| Compound | % (by weight) |
|---|---|
| Silica (SiO₂) | 71 |
| Sodium Oxide (Na₂O) | 14 |
| Potassium Oxide (K₂O) | 1 |
| Calcium Oxide (CaO) | 9 |
| Alumina (Al₂O₃) | 3 |
| Magnesium Oxide (MgO) | 2 |

In this embodiment, the expanded glass granule 80 is a manufactured and shaped granule having a generally spherical shape. The expanded glass granule 80 may be sized between 4 mm and 100 mm. However, preferably it measures between 6 mm and 20 mm. In other embodiments, differently shaped granules could also be used to similar advantage. The expanded glass granulate product made commercially available by Dennert Poraver GmbH of Schlüsselfeld, Germany under the name PORAVER™ has been found to be suitable for use as the expanded glass granule. This product is manufactured from recycled glass and has been used in the past as a component of building materials such as plasters, mortars, adhesives and fillers. However, it will be appreciated that other granulate products exhibiting similar material properties and having different chemical compositions could also be employed to advantage.

The expanded glass granules 80 are randomly packed in the packing bed 24 to permit the configuration of the packing material 30 to be optimized for the particular shape of the packing bed 24. In this manner, improved packing efficiency may be achieved thereby leading to a more uniform distribution of the waste gas streams and the trickling liquid in the packing material 30. As a result, problems associated with gas channelling within the packing bed may be mitigated.

The relatively light-weight/low density characteristics of the packing material 30 tend to facilitate handling of the packing material when charging and discharging the packing material 30 in the packing bed 24 and during maintenance and servicing operations. In particular, the packing material 30 may be removed from the packing bed 24 to permit the excess biomass collected on the surface of the packing material to be washed off thereby allowing recycling of the packing material. In this way, the clogging problems typically associated with conventional biotrickling filter packing materials tend to be mitigated in the packing material 30. Alternatively, the packing material 30 may be easily removed from the packing bed for replacement using a vacuum device.

In addition, freight costs associated with the packing material tend to be lower than those associated with the heavier conventional biotrickling filter media thereby enhancing the cost effectiveness of the packing material 30.

Operation

The operation of the biotrickling filter system 20 will now be described in greater detail. The biotrickling filter system 20 is supplied with a waste gas stream from, for example, a wastewater treatment plant or a rendering plant. The contaminated air is drawn into the housing 22 through the waste gas inlet 38 and is urged to flow upwardly through the packing bed 24 by the operation of the blower 36. The recirculating pump 56 draws liquid from the sump 25, which liquid is sprayed onto the surface of the packing material 30 by the nozzles 64 of the liquid distribution means 60.

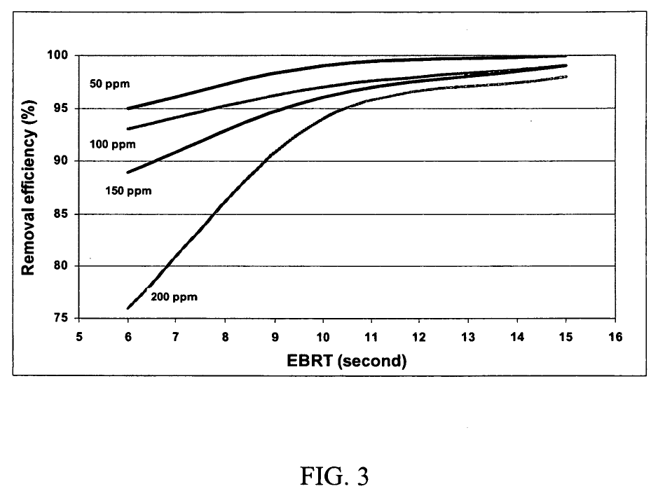

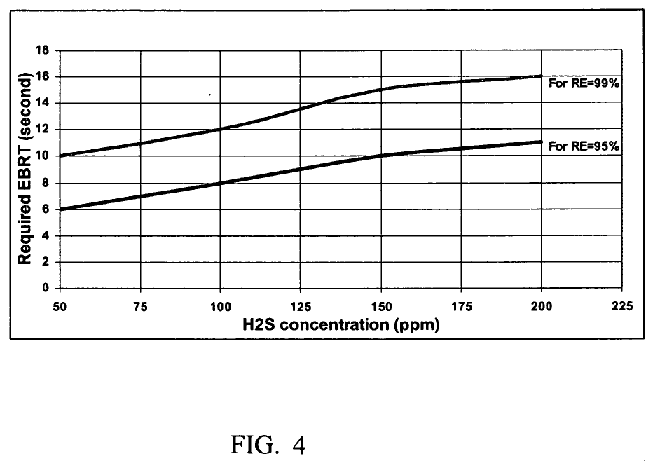

As the waste gas stream flows upwardly and the liquid trickles downwardly through the packing material 30, the contaminants are solubilized in the trickling liquid and undergo phase transfer from the gas phase to the liquid phase. In the biotrickling filter system of the present embodiment, the phase transfer of hydrogen sulfide tends to occur more rapidly in the packing material 30 than in conventional biotrickling filter media It is believed that the higher rate of phase transfer of hydrogen sulfide is due to its particular affinity for the expanded glass granule 80. This increased affinity for the expanded glass granule 80 may allow the biotrickling filter system 20 to achieve higher removal efficiencies (elimination capacities) for hydrogen sulfide than were previously obtained with biotrickling filter systems employing conventional packing material. An example of the removal efficiencies achieved for hydrogen sulfide (at various concentrations and at various EBRTs) using the biotrickling filter system having the packing material provided in accordance with an embodiment of the present invention, are shown in FIGS. 3 and 4.

Once the contaminants have transitioned to the liquid phase, the contaminants are adsorbed onto the biofilm formed on the surface of the expanded glass granule 80 and then degraded by the metabolic activities of the microorganisms. Carbon dioxide and water are produced as a result of the biological oxidation of VOCs. The sulfur-based compounds may break down into sulfites (SO₃²⁻), sulfates (SO₄²⁻) or sulfur (S). The water soluble sulfur compounds tend to be flushed out of the packing bed 24 by the recirculating trickling liquid. Once the contaminants are removed, the treated air stream is exhausted from the housing 22 through the outlet 42.

The coarse granular configuration of the expanded glass granule 80 as well as its characteristic low density/light weight tends to permit easy washing of the packing material to remove not only the products of the contaminant degradation but also any excessive biomass which may have accumulated on the surface of the packing material 30. The problems associated with high gas flow resistance and clogging encountered in known biotrickling filter media tend to be minimized in the packing material 30. Accordingly, the packing material may be recycled, regenerated and reused with relative ease thus tending to impart to it a relatively long service life.

The residue trickling liquid from the packing bed 24 collects in the sump 25. Effluent and nutrients may be drawn from tanks 44 and 46 and pumped into the sump 25 wherein they mix with the residue trickling liquid. The mixture is conveyed back to the liquid recirculation system 56 for reuse. Occasionally, a portion of the liquid along with small amounts of biomass and dissolved pollutant are discharged or purged from the sump 25 through the drain line 72.

During operation of the biotrickling filter system 20, the control system 74 monitors the operational parameters of the system to ensure the optimal operating conditions are maintained within the packing bed 24. For instance, if the pH value measured by the pH sensor 68 falls outside of the desired range, some of the trickling fluid could be purged and effluent or potable water could be added through the liquid recirculation system 56 to adjust the pH. Alternatively, the pH may be adjusted chemically with the addition of a liquid buffer. In another example, if the liquid level sensor 52 detects that the liquid level in the sump 25 is too high or too low, the control system 74 may cause the appropriate remedial action to be taken (i.e. excess liquid may be purged through the drain line 72 or the sump 25 may be recharged with liquid from the effluent tank 44).

With its light weight/low density characteristics, the packing material 30 allows for greater flexibility in the design of biotrickling filter systems. More specifically, the packing material 30 can be used to lighten the overall weight of a biotrickling filter system thereby lessening the need for more structural support (i.e. larger and heavier foundations). In addition, in biotrickling filter systems that employ the packing material 30, the height of the column in the packing bed may be increased to permit greater bed depth and higher inlet gas velocities. This may allow the installation footprint of the biotrickling filter system to be reduced for even greater versatility.

The removal kinetics of various contaminants using a biotrickling filter system having the packing material 30 in accordance with an embodiment of the present invention have been examined through performance data obtained in laboratory during initial pilot studies. The findings obtained from the different studies are described as follows:

Using a biotrickling filter system constructed and operated in accordance with the principles of the present invention, high H₂S removal efficiency at high inlet concentrations in low empty bed residence times (EBRT) has been consistently obtained. More specifically, the biotrickling filter system has achieved 93% removal of 100 ppm of H₂S in 6 seconds EBRT and 99% removal in 12 seconds EBRT. At higher concentrations of H₂S, the biotrickling filter system tended to perform very well. The biotrickling filter system successfully removed greater than 99% of 200 ppm of H₂S in 16 seconds EBRT. In comparison, the high performance polyurethane foam media currently used by the assignee of the present application, BIOREM Technologies Inc. of Guelph, Ontario in its biotrickling filter systems made commercially available under the name MYTILUS™, is capable of removing only 81% of 100 ppm of H₂S in 6 seconds EBRT and 99% in 15 seconds EBRT. At a concentration of 200 ppm of H₂S, the polyurethane foam packing material used in the MYTILUS™ biotrickling filter system was able to achieve only 75% removal efficiency.

Performance data for the removal of H₂S at concentrations of up to 200 ppm with the biotrickling filter packing media provided in accordance with the principles of the present embodiment (identified as “LWE”) and with the known packing material (identified as “PUF”) currently used in the MYTILUS™ biotrickling filter system are compared in Table 1 below:
Table 1. Removal Efficiencies of H₂S using the biotrickling filter packing material (LWE) provided in accordance with the principles of the present invention and the known packing material (UF) currently used in the MYTILUS™ biotrickling filter system

| | | |
|---|---|---|
| | | Removal |
| | | efficiencies |
| H₂S | EBRT | (%) |

| concentration (ppm) | (seconds) | LWE | PUF |
|---|---|---|---|
| 50 | 15 | 100 | 99 |
| | 10 | 99 | 97 |
| | 6 | 95 | 90 |
| 100 | 15 | 99 | 99 |
| | 10 | 97 | 96 |
| | 6 | 93 | 81 |
| 150 | 15 | 99 | 99 |
| | 10 | 96 | 93 |
| | 6 | 89 | 80 |
| 200 | 15 | 98 | 90 |
| | 10 | 94 | 75 |

As will be appreciated, the biotrickling filter packing material provided in accordance with the principles of the present invention exhibits improved removal efficiencies.

Using the biotrickling filter system constructed and operated in accordance with the principles of the present invention, an elimination capacity of greater than 100 g/m³/h has been obtained for hydrogen sulfide.

It will thus be appreciated that the physical, material and biological characteristics of the packing material 30 as described above enable the packing material to perform better than other known packing materials. Whereas some conventional packing materials are able to achieve satisfactory removal rates for hydrogen sulfide by improving biodegradation of the contaminants, the packing material 30 is designed to encourage both phase transfer and enhance biodegradation of the contaminants. As a result, the packing material is able to remove hydrogen sulfide from waste gas streams with superior efficiency.

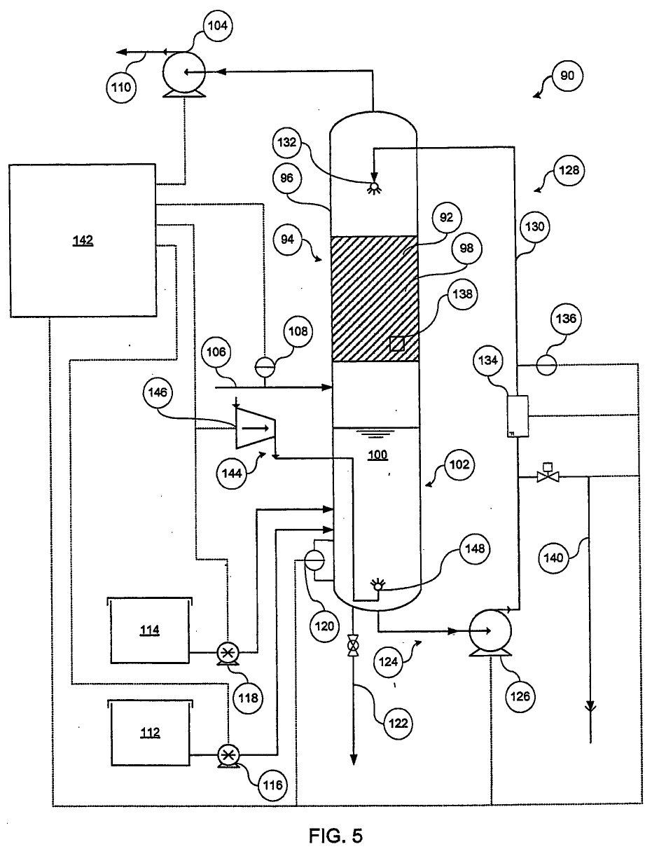

In the foregoing embodiment, use of the expanded glass granules 80 as the packing material in a biotrickling filter, was described. However, it will be appreciated that, due to its particular physical, material and biological characteristics (described above), the expanded glass granule 80 may also be suitable for use in a bioscrubbing filter system. Referring to FIG. 5, there is shown a bioscrubber 90 that employs the expanded glass granules 80 as packing material 92 in its air phase reactor 94.

The bioscrubber 90 is generally similar to the biotrickling filter system 20 in that it includes a housing 96 that encloses a packing bed 98 and a sump or liquid reservoir 100. However, in this embodiment, the size of the sump 100 has been substantially increased to create a liquid phase reactor 102 in which suspended microbial cultures may biodegrade the solubilized contaminants.

The packing bed 98 is generally similar in structure and construction to the packing bed 24 of the biotrickling filter system 20 except that in the bioscrubber 90, the packing bed 98 is sized smaller than the packing bed 24. The packing bed 98 defines the air phase reactor 94 in which the contaminants undergo mass transfer from the gas to the liquid phase. As in the biotrickling filter system 20, a plurality of expanded glass granules 80 are randomly packed in the packing bed 98. While expanded glass granules 80 measuring between 4 mm and 100 mm may be used in the packing bed 98, preferably, the size of the granules 80 is in the range of 6 mm to 20 mm.

A blower or fan 104 is provided to draw the contaminated air stream from a waste gas inlet 106 and through the packing bed 98. A gas flow sensor 108 positioned within the waste gas inlet 106 measures the flow of contaminated air entering the bioscrubber 90.

In this embodiment, the bioscrubber 90 is operated as a counter-current system with the contaminated air stream being urged to flow upward through the packing bed 98 in a direction opposite to that of the trickling liquid. It will, however, be appreciated that in an alternative embodiment, the bioscrubber could operate as a co-current system.

An outlet 110 located near the top of the housing 96 permits a cleaned air stream to exit the housing 96 following treatment in the packing bed 98. Optionally, a mist eliminator (not shown) positioned upstream of the outlet 110 could also be provided to remove fine liquid droplets from the cleaned air stream.

The sump 100 holds the trickling liquid which will be used in the bioscrubber 90 and contains suspended growth microbial cultures. The trickling liquid may include effluent and/or freshwater and recirculated water and may be supplemented with a nutrient solution containing minerals such as nitrogen, phosphorus and potassium, and/or other additives such as liquid buffers for adjusting the pH of the packing material 92. In this embodiment, the effluent and nutrient solution are drawn into the sump 100 from dedicated effluent tank 112 and nutrient tank 114 using pumps 116 and 118, respectively. The effluent may be partially treated wastewater containing some residual nutrients. In other embodiments, the effluent may be provided by an effluent supply line connected to a wastewater treatment plant. A liquid level sensor 120 measures the level of the liquid in the sump 100. Some or all of the trickling fluid may be drained from the sump 100 through a first manually actuated drain line 122. Aeration means 144 are operable to deliver oxygen to the liquid stored in the sump to encourage aerobic biodegradation of the contaminants within the liquid phase reactor 94. The aeration means 144 includes an air compressor 146 operatively connected to an air diffuser 148 located within the sump 100.

The bioscrubber 90 further includes a liquid recirculation system 124 that is generally similar to the liquid recirculation system 56 of the biotrickling filter system 20. It includes a recirculation pump 126 and liquid distribution means 128. The recirculation pump 126 is operable to draw from the sump 100 the trickling liquid for delivery to the packing bed 98. The liquid distribution means 128 includes a feed line 130 with a plurality of nozzles 132 for continuously or intermittently spraying/trickling liquid onto the packing material 92. A liquid flow sensor 134 and a pH sensor 136 measure the flow rate and pH of the trickling liquid flowing through the feed line 130. Also provided, is a temperature sensor 138 to monitor the temperature of the packing material. Other sensors may also be provided, for instance, to monitor the need for additional nutrients. The liquid recirculation system 124 has a second drain line 140 for periodically purging the trickling fluid.

A control system 142 generally similar to control system 74 governs the operation of the bioscrubber 90. The control system 142 communicates with (i.e. receives input signals from, and transmits output signals to,) the various sensors 108, 120, 134, 136 and 138 and other system equipment and can actuate the blower 104, the pumps 116, 118 and 126, the drain lines 122 and 140 and the liquid recirculation system 124 to ensure proper operation of the system.

The operation of the bioscrubber 90 will now be described in greater detail. The bioscrubber 90 is supplied with a waste gas stream from, for example, a wastewater treatment plant or a rendering plant. The contaminated air is drawn into the housing 96 through the waste gas inlet 106 and is urged to flow upwardly through the packing bed 98 by the operation of the blower 104. The recirculating pump 126 draws liquid from the sump 100, which liquid is sprayed onto the surface of the packing material 92 by the nozzles 132 of the liquid distribution means 128.

As the waste gas stream flows upwardly and the liquid trickles downwardly through the packing material 92, the contaminants are solubilized in the trickling liquid and undergo phase transfer from the gas phase to the liquid phase. In the bioscrubber of the present embodiment, the phase transfer of hydrogen sulfide will tend to occur more rapidly in the packing material 92 than in conventional bioscrubber media. It is believed that the higher rate of phase transfer of hydrogen sulfide is due to its particular affinity for the expanded glass granule 80. This increased affinity for the expanded glass granule 80 may allow the bioscrubber to achieve higher removal efficiencies (elimination capacities) for hydrogen sulfide than were previously obtained with bioscrubbers employing conventional packing material.

Once the contaminants have transitioned to the liquid phase, the contaminants are absorbed in the trickling liquid flowing through the air phase reactor 94. The trickling fluid exiting the packing bed 98 is collected in the liquid phase reactor 102 (sump 100) wherein microorganisms suspended in the liquid biodegrade the contaminants.

Carbon dioxide and water are produced as a result of the biological oxidation of VOCs. The sulfur-based compounds may break down into sulfites (SO₃²⁻), sulfates (SO₄²⁻) or sulfur (S). The water soluble sulfur compounds tend to be flushed out of the packing bed 98 by the recirculating trickling liquid. Once the contaminants are removed, the treated air stream is exhausted from the housing 96 through the outlet 110.

The residue trickling liquid from the packing bed 98 collects in the sump 100. Effluent and nutrients may be drawn from tanks 112 and 114 and pumped into the sump reservoir 100 wherein they mix with the residue trickling liquid. The mixture is conveyed back to the liquid recirculation system 124 for reuse. Occasionally, a portion of the liquid along with small amounts of biomass and dissolved pollutant are discharged or purged from the sump reservoir 100 through first or second drain lines 122, 140.

During operation of the biotrickling filter system 20, the control system 142 monitors the operational parameters of the system to ensure the optimal operating conditions are maintained within the packing bed 98. For instance, if the pH value measured by the pH sensor 136 falls outside the desired range, an appropriate chemical solution, such as a liquid buffer, may be added through the liquid recirculation system 124. In another example, if the liquid level sensor 120 detects that the liquid level in the sump 100 is too high or too low, the control system 142 may cause the appropriate remedial action to be taken (i.e. excess liquid may be purged through the drain line 122 or the sump 100 may be recharged with liquid from the effluent tank 112 or effluent supply line).

Although the foregoing description and accompanying drawings relate to specific preferred embodiments of the present invention as presently contemplated by the inventor(s), it will be understood that various changes, modifications and adaptations, may be made without departing from the spirit of the invention.

What is claimed is:

1. A method for removing odour causing compounds from a waste gas stream, the method comprising:
providing a biotrickling filter system having a packing material, the packing material including a plurality of expanded glass granules;
urging the waste gas stream to flow through the packing material of the biotrickling filter system;
urging a liquid to trickle through the packing material; and
collecting the trickling liquid in a sump and recirculating the trickling liquid from the sump into the biotrickling filter system for reuse.

2. The method of claim 1 wherein the expanded glass granules are composed primarily of silica and alkali oxides.

3. The method of claim 2 wherein the alkali oxides are predominantly sodium oxide.

4. The method of claim 1 wherein each expanded glass granule measures between 4 mm and 100 mm.

5. The method of claim 4 wherein each expanded glass granule measures between 6 mm and 20 mm.

6. The method of claim 1 wherein each expanded glass granule has a generally spherical shape.

7. The method of claim 1 wherein urging the waste gas stream to flow through the packing material of the biotrickling filter system, includes forcing the waste gas stream to flow downwardly through the packing material.

8. The method of claim 1 wherein urging the waste gas stream to flow through the packing material of the biotrickling filter system, includes forcing the waste gas stream to flow upwardly through the packing material.

9. The method of claim 1 wherein the flow of the waste gas stream and the flow of the trickling liquid are one of counter-current and co-current.

10. The method of claim 1 wherein urging a liquid to trickle through the packing material includes, distributing the liquid onto the surface of the packing material.

11. The method of claim 1 wherein the trickling liquid includes effluent and recirculated trickling liquid.

12. The method of claim 1 wherein the trickling liquid further includes a nutrient solution containing at least one of phosphorus, nitrogen and potassium.

13. The method of claim 1 wherein the odour causing compounds include hydrogen sulfide and volatile organic compounds.

14. A biotrickling filter system comprising:
a housing;
an inlet connected to the housing for receiving contaminated air;
an outlet connected to the housing for exhausting cleaned air;
a packing bed located within the housing between the inlet and the outlet, the packing bed having a packing material through which the contaminated air is forced to flow, the packing material including a plurality of expanded glass granules;
means for drawing the contaminated air from the inlet into the packing bed and through the packing material;
means for distributing a liquid onto the surface of the packing material for trickling therethrough;
a sump disposed below the packing bed for collecting the trickling liquid; and
means for recirculating the trickling fluid through the biotrickling filter, the liquid recirculating means being operatively connected to the sump and the liquid distributing means.

15. The biotrickling filter system of claim 14 wherein the expanded glass granules are composed primarily of silica and alkali oxides.

16. The biotrickling filter system of claim 14 wherein the alkali oxides are predominantly sodium oxide.

17. The biotrickling filter system of claim 14 wherein each expanded glass granule measures between 4 mm and 100 mm.

18. The biotrickling filter system of claim 17 wherein each expanded glass granule measures between 6 mm and 20 mm.

19. The biotrickling filter system of claim 14 wherein each expanded glass granule has a generally spherical shape.

20. A method for removing odour causing compounds from a waste gas stream, the method comprising:
providing a bioscrubber having a packing material, the packing material including a plurality of expanded glass granules;
urging the waste gas stream to flow through the packing material of the bioscrubber;
urging a liquid to trickle through the packing material; and
collecting the trickling liquid in a sump and recirculating the trickling liquid from the sump into the bioscrubber for reuse.

21. The method of claim 20 wherein the odour causing compounds include hydrogen sulfide and volatile organic compounds.

22. A bioscrubber comprising:
a housing;
an inlet connected to the housing for receiving contaminated air,
an outlet connected to the housing for exhausting cleaned air; and
a packing bed located within the housing between the inlet and the outlet, the packing bed having a packing material through which the contaminated air is forced to flow, the packing bed defining an air phase reactor wherein contaminants in the contaminated air may undergo phase transfer from the gas phase to the liquid phase, the packing material including a plurality of expanded glass granules;
means for drawing the contaminated air from the inlet into the packing bed and through the packing material;
means for distributing a liquid onto the surface of the packing material for trickling therethrough;
a sump disposed below the packing bed for collecting the trickling liquid, the sump defining a liquid phase reactor wherein contaminants in the tricking fluid are biodegraded by suspended microbial cultures; and
means for recirculating the trickling fluid through the biotrickling filter, the liquid recirculating means being operatively connected to the sump and the liquid distributing means.
